(19) United States
(12) Patent Application Publication
(10) Pub. No.: US 2011/0106085 A1
(43) Pub. Date: May 5, 2011

(54) ADJUSTABLE OCCIPITAL VERTEBRAL FIXATION SYSTEM

(75) Inventors: William Barry Null, Olive Branch, MS (US); Marc Thomas Paul, Memphis, TN (US); Benjamin David Cowan, Memphis, TN (US); Jayant Jangra, Memphis, TN (US)

(73) Assignee: Warsaw Orthopedic, Inc., Warsaw, IN (US)

(21) Appl. No.: 12/609,687

(22) Filed: Oct. 30, 2009

(51) Int. Cl. A61B 17/80

(52) U.S. Cl. 606/70

(57) ABSTRACT

An occipital plating system includes a plating assembly with a plate body defining a profile adapted for positioning on the occiput. The plate body includes a central portion that extends bi-laterally across the central axis of the spinal column to opposite lateral portions of the plate. At least one coupling member extends from each of the lateral portions of the plate body to receive a connecting element extending from the spinal column.

### BACKGROUND

Various devices have been employed for fixation of the occiput with the cervical vertebrae. Posterior systems include a plate attached to the occiput with screw fixation. A rod extends from the plate and along the cervical vertebrae for attachment to the cervical vertebrae to provide a platform for fixation. Spanning of single or multiple levels of the cervical spine results in fixation of these levels in conjunction with the occiput.

In the cervical region, anatomical variations make it difficult to provide a plate that adequately fits with the anatomy of the occiput. Furthermore, the occipital bone or skull has variations in bone density and thickness so that bone screw purchase in some areas is less than desired to secure the plate to the bone. Systems for occipital and cervical stabilization are needed that provide adequate stabilization while providing an optimal or desired fit with the occiput and vertebral anatomy. Such systems should further reduce the invasiveness and complexity of the procedure for the surgeon and reduce operating room time for the patient.

### SUMMARY

An occipital plating system includes a plating assembly with a plate body defining a profile adapted for positioning on the occiput. The plate body includes a central portion that extends bi-laterally across the central axis of the spinal column to opposite lateral portions of the plate. At least one coupling member extends from each of the lateral portions of the plate body to receive a connecting element extending from the spinal column.

According to one aspect, the plate body includes a window extending therethrough that opens at upper and lower surfaces of the plate body. The window includes a central leg extending along the central portion of the plate body, and lateral legs that extend along the lateral portions of the plate body. In one embodiment, the plate body includes a central projecting portion extending from the central portion along the central axis between the lateral portions, and the window includes a middle leg through the middle projecting portion. The legs of the window are connected and continuous with one another so that the window forms a single, common opening through the plate body. The window is sized relative to a fastener so that when the fastener is positioned through the window to engage the plate body to the occiput the fastener includes a head that abuts the plate body adjacent the window. One or more fasteners can be positioned at any position along the window to secure the plate body to the bone.

According to another aspect, the plate body includes a plurality of thin slots or grooves in the central portion and lateral portions that facilitate flexing or contouring of these portions of the plate body in multiple directions relative to the medial-lateral axis and central axis. The slots include an open end at one of the opposite inner and outer edges of the plate body and a terminal end located within the plate body between the inner and outer edges. The slots include a serpentine or sinusoidal shape or any suitable alternating configuration between the terminal end and the open end. Adjacent slots are alternated so that one of the adjacent slots includes an open end at the inner edge of the plate body and the other of the adjacent slots includes its open end at an outer edge of the plate body. In addition, the peaks of the alternating shape of one of the adjacent slots are aligned to maintain a desired amount of plate material between slots. The slots are configured to permit bending and flexing of the plate body while maintaining structural integrity of the plate body to carry out its spinal and occipital stabilization functions.

According to another aspect, each of the lateral portions of the plate body is pivotally or rotationally coupled to an opposite end of the central portion of the plate body with respective ones of first and second coupling mechanisms at the opposite ends of the central portion. The coupling mechanisms each establish a predefined pivot or rotation axis that permits the plate body to be contoured to fit the anatomy without bending the plate body. In a further embodiment, the lateral portions of the plate body are translationally coupled to the central portion of the plate body so that the lateral portions are independently movable relative to the central portion and to one another in a direction toward and away from the central axis.

These and other aspects will also be apparent from the following description and the Figures.

### BRIEF DESCRIPTION OF THE DRAWINGS

FIG. 1 shows an occipital vertebral fixation system looking cephaladly toward the occiput.

FIG. 2 is a perspective view of a plate assembly of the system of FIG. 1.

FIG. 3 is a perspective of another embodiment plate assembly.

FIG. 4 is a perspective view of another embodiment plate assembly.

FIG. 5 is a perspective view of another embodiment plate assembly.

### DESCRIPTION OF THE ILLUSTRATED EMBODIMENTS

For the purpose of promoting an understanding of the principles of the invention, reference will now be made to the illustrated embodiments thereof and specific language will be used to describe the same. It will nevertheless be understood that no limitation of the scope of the invention is thereby intended. Any such alterations and further modifications in the invention, and any such further applications of the principles of the invention as described herein are contemplated as would normally occur to one skilled in the art to which the invention relates.

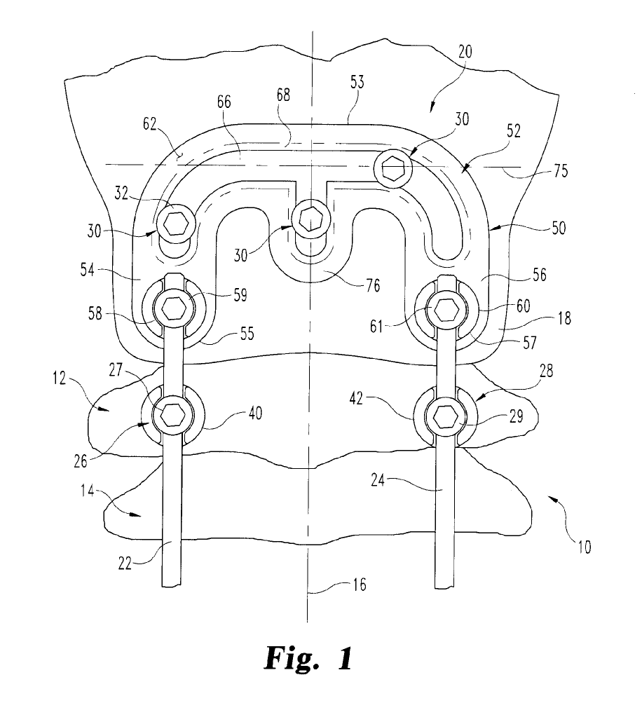

Referring to FIG. 1, there is shown a posterior elevation view of a spinal column segment 10 including the upper cervical vertebra C1 designated at 12 and the next lower cervical vertebra C2 designated at 14. Cervical vertebrae 12, 14 extend along the central axis 16 of spinal column segment 10. Occiput 18 resides at the superior end of C1 vertebra 12. Occipital fixation system 20 includes an occipital plate assembly 50 engaged to occiput 18 and a pair of elongated connecting elements 22, 24 engaged to at least one of the cervical vertebrae 12, 14. Connecting elements 22, 24 are bi-laterally positioned along opposite sides of the spinous processes of one or more of the cervical vertebrae 12, 14 and central axis 16 of the spinal column, and engaged to posterior spinal elements on one or more of cervical vertebrae 12, 14 with one or more anchors 26, 28, respectively. One or both of connecting elements 22, 24 may further extend along the spinal column to one or more of the thoracic, lumbar and sacral regions and engaged to one or more vertebrae thereof with any suitable connector.

Connecting elements 22, 24 extend along the spinal column and can be rigid to prevent motion, flexible, or partially flexible to allow at least limited motion in the stabilized motion segments. Connecting elements 22, 24 can be in the form of rods, plates, bars, tethers, cables, dampeners, springs, or other structure to provide a desired stabilization effect. The connecting elements 22, 24 can be secured to the vertebrae with any suitable bone anchoring device. In the illustrated embodiment, bone anchors 26, 28 are engaged to vertebra 12 and also to connecting elements 22, 24 with engaging members 27, 29 engaged to the respective bone anchors 26, 28.

The bone anchors 26, 28 can include bone screws with a receiving portion 40, 42, respectively, for receiving the respective connecting element 22, 24. The receiving portion 40, 42 can be movable relative to the bone engaging portion or fixed relative to the bone engaging portion of the respective anchoring device 26, 28. The receiving portions 40, 42 can be in the form of a top-loading, side-loading or bottom loading member with a channel to receive the respective connecting element 22, 24. Engaging members 27, 29 can be provided in the form of a set screw, nut, cap, slide-lock device, or other device can be engaged to the respective receiving portion 40, 42 to secure the connecting element 22, 24 in the respective receiving portion 40, 42. The receiving portions 40, 42 can also be in the form of a clamp, wire or other device positioned around the connecting element 22, 24 to secure it to the bone anchoring portion. The bone anchoring portion (not shown) can be in the form of a bone screw, hook, cable, anchor, staple, interbody device, clamp, or other suitable bone engaging structure to secure the respective connecting element 22, 24 to the corresponding bony structure.

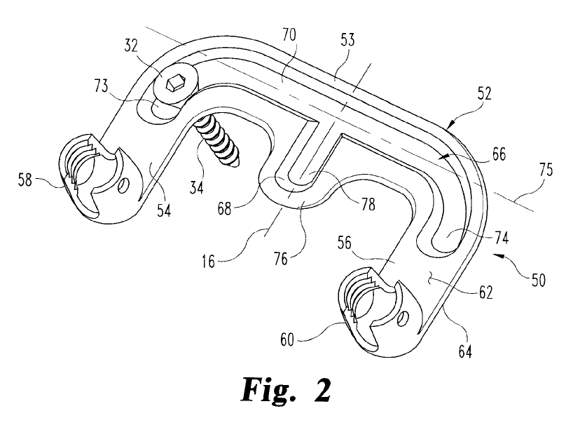

Plate assembly 50 secures at least one of the connecting elements 22, 24 to the occiput 18. Plate assembly 50 includes a plate body 52 engaged to occiput 18 with one or more fasteners 30. As also shown in FIG. 2, plate body 52 includes a central portion 53 that extends along a medial-lateral axis 75 and orthogonally to a central axis that is aligned with the central axis 16 of the spinal column between opposite lateral portions 54, 56. Lateral portions 54, 56 are located and offset bi-laterally from central axis 16 and extend caudally from medial-lateral axis 75 so that coupling member 58, 60 are positioned on opposite sides of the medial nuchal line. Lateral portions 54, 56 extend caudally from central portion 53 in a direction generally along central axis 16 to a respective one of the outer ends 55, 57 of plate body 52.

The laterally and caudally located outer ends 55, 57 each include a respective one of coupling members 58, 60, which extend proximally away from the occiput 18 from the respective lateral portion 54, 56 to receive the respective connecting element 22, 24 therein. An engaging member 59, 61, such as a set screw, cap, nut or other device can be engaged to each of the coupling members 58, 60 to secure the respective connecting element 22, 24 to, in, on or around coupling members 58, 60. Alternatively, coupling members 58, 60 can be configured to crimp, clamp, frictionally lock, capture or otherwise secure the respective connecting element 22, 24 thereto without a separate engaging member. In the illustrated embodiment, coupling members 58, 60 are fixed relative to plate body 52 and are formed as one piece with plate body 52. Other embodiments contemplate that coupling members 58, 60 are separate from plate body 52, and are fixed, movable or rotatable relative to plate body 52. Any one or combination of rotatable, pivotable, or translation of one or both coupling members 58, 60 along central axis 16 and/or medial-lateral axis 75 relative to plate body 52 is contemplated.

Plate body 52 includes an upper or proximally-directed surface 62 and an opposite lower or distally directed surface 64. A window 66 is provided through plate body 52 that opens at upper and lower surfaces 62, 64. Window 66 is surrounded by a recessed surface 68 in plate body 52. Recessed surface 68 extends completely around window 66 and forms a well or depression that receives a head 32 of a fastener 30 positioned in window 66 so that head 32 of fastener 30 is located at least partially in window 66 and recessed more distally relative to upper surface 62 than if recessed surface 68 were not provided, reducing the profile or height of fastener 30 extending proximally from plate body 52. Other embodiments contemplate that recessed surface 68 is formed by scallops or a series of recessed locations in plate body 52.

Window 66 includes a center leg 70 elongated along axis 75 that extends through central portion 53 of plate body 52, and lateral legs 72, 74 that are elongated and extend through lateral end portions 54, 56, respectively, of plate body 52. Lateral legs 72, 74 are located at and extend from a respective end of center leg 70. In addition, plate body 52 includes a central projecting portion 76 extending from central portion 53 along central axis 16 in a caudal direction. Window 66 includes a middle leg 78 through central projecting portion 74. In the illustrated embodiment, window 66 is continuous and uninterrupted between center leg 70, lateral legs 72, 74 and middle leg 78. Plate body 52 includes an M-shape with the legs of the M-shape extending along central axis 16 in the caudal direction, and window 66 includes a similar M-shape that is completely surrounded by plate body 52. Other embodiments contemplate other shapes for plate body 52 and window 66, including U-shapes, V-shapes, W-shapes, C-shapes, E-shapes, Y-shapes, K-shapes, butterfly shapes, and square or rectangular bracket type shapes, for example.

Window 66 and its continuous configuration through the central, lateral and middle portions of plate body 52 provides the surgeon freedom to locate fasteners 30 at any location in window 66 that provides the desired engagement with the underlying anatomy. For example, fasteners 30 can be targeted to areas where the bone has the desirable density and thickness for optimal engagement of plate body 52 to occiput 18. Fasteners 30 can be moved or positioned along window 66 and any one or all of its legs 70, 72, 74, 78 to infinitely vary the spacing between fasteners 30. The surgeon can also select any number of fasteners 30 for positioning through window 66. Window 66 improves visibility when driving or engaging fasteners 30 to the bone so that the bone and fastener interface can be directly observed through window 66. Bone graft or other bone material can be placed through window 66 to facilitate strengthening of the underlying bone.

Fasteners 30 can be in the form of bone screws with a threaded shaft 34 sized for positioning through window 66 and a proximal head 32 that is positioned against a proximally oriented surface of plate body 52 to secure it against or adjacent to occiput 18. Recess 68 and/or upper surface 62 of plate body 52 can include grooves, knurlings or other features to interlock with head 32 when head 32 is positioned flushed thereagainst. Fastener 32 may also include a washer or other feature to seat against and secure fastener 32 with plate body 52. In the illustrated embodiment, three fasteners 30 are shown and plate body 52 includes a single window 66 to accommodate respective ones of the three fasteners. It is contemplated that fewer than three fasteners may be provided so that one or more legs of window 66 do not include a fastener. Furthermore, more than three fasteners 30 may be provided so that each of legs 70, 72, 74, 78 includes at least one fastener 30, and/or that one or more of legs 70, 72, 74, 78 include multiple fasteners 30. Fasteners 30 can be provided and inserted through all or a portion of the legs 70, 72, 74, 78 in plate body 52. The appropriate number of fasteners 30 can be selected based on the desired level or amount of fixation of plate body 52 to occiput 18.

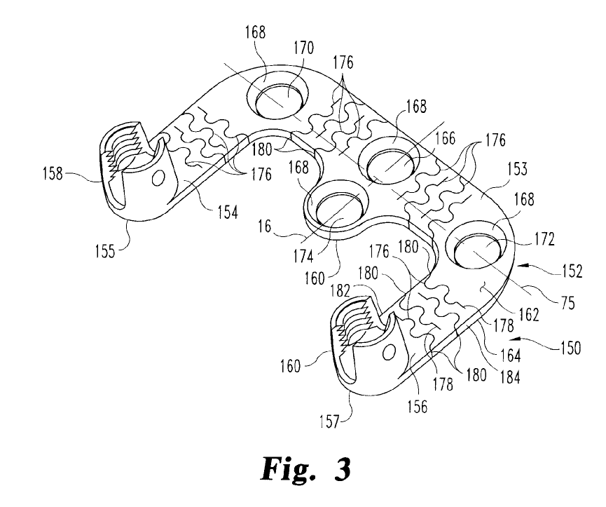

Referring to FIG. 3, there is shown another plate assembly embodiment designated at 150 that is positionable along the occiput 18 in a manner similar to that discussed above with respect to plate assembly 50 discussed above. Plate assembly 150 includes plate body 152 with coupling members 158, 160 that can include any of the features of coupling members 58, 60 discussed above with respect to plate assembly 50 to engage connecting elements 22, 24. Plate body 152 includes a central portion 153 that extends transversely to the central axis 16 and along medial-lateral axis 75 to opposite lateral portions 154, 156. Lateral portions 154, 156 are located bi-laterally relative to central axis 16 and extend caudally from medial-lateral axis 75 to laterally offset coupling members 158, 160 to opposite sides of the medial nuchal line. Lateral portions 154, 156 extend caudally in a direction generally along central axis 16 to a respective one of the outer ends 155, 157. In addition, plate body 152 includes a central projecting portion 160 extending from central portion 153 along central axis 16 in a caudal direction. Plate body 152 includes a generally M-shaped configuration with the legs of the M oriented in the caudal direction when implanted. Other embodiments contemplate other shapes for plate body 152, including U-shapes, V-shapes, W-shapes, C-shapes, E-shapes, Y-shapes, K-shapes, butterfly shapes, and square or rectangular bracket type shapes, for example.

Plate body 152 includes an upper or proximally-directed surface 162 and an opposite lower or distally directed surface 164. A number of holes, slots or windows 166, 170, 172, 174 are provided through plate body 152, and each hole opens at upper and lower surfaces 162, 164. Each of holes 166, 170, 172, 174 is surrounded by a recessed surface 168 in plate body 152. Recessed surface 168 extends completely around the respective hole and receives a fastener 30 positioned in the respective hole so that the head 32 of fastener 30 is located at least partially in the respective hole and recessed more distally relative to upper surface 162 than if recessed surface 168 were not provided to reduce the profile of fasteners 30 extending from upper surface 162.

Plate body 152 includes a number of slots 176 formed in plate body 152 to enhance flexibility of plate body 152 and facilitate contouring of plate body 152 to fit along the occiput 18. Slots 176 allow plate body 152 to be contoured in multiple directions relative to central axis 16 and medial-lateral axis 75 to fit the occipital anatomy and maintain a low profile that conforms to the natural anatomy of the occiput and skull and also to provide a smoother profile after implantation. In the illustrated embodiment, four slots 176 are located in lateral portion 154, four slots 176 are located in lateral portion 156, and eight slots 176 are located in central portion 153 with four slots on each side of central axis 16 and hole 66. Hole 170 is located between the slots of lateral portion 154 and the slots of central portion 153, and hole 172 is located between the slots of lateral portion 156 and the slots of central portion 153.

Slots 176 occupy a major portion of the length of each of the lateral portions 154, 156 and of central portion 153 to maximize flexibility and contourability, while the shape and configuration of slots 176 maintains the structural integrity of plate body 152. For example, the spacing between the most-cephalad and most-caudal slots 176 of lateral portions 154, 156 occupies a majority of the length of the lateral portions 154, 156, and the spacing between the most-lateral slots 176 of central portion 153 occupies a majority of the length of central portion 153. The slots 176 in lateral portions 154, 156 extend generally along medial-lateral axis 75 and facilitate bending and contouring of plate body 152 around medial lateral axis 75, and slots 176 in central portion 153 extend generally along central axis 16 and facilitate bending and contouring of plate body 152 around central axis 16. Slots 176 include a sinusoidal, serpentine or otherwise alternating shape along their respective lengths.

The alternating shape of slots 176 facilitates non-linear and non-angular bending and contouring of plate body 152 for optimal fit. In the illustrated embodiment, the peaks of the sinusoidal shape of one of slots 176 generally aligns with respective ones of the peaks of the sinusoidal shape of an adjacent slot to maintain a desired spacing between slots and an appropriate amount of plate material between adjacent slots 176 for structural integrity. Each of the slots 176 is formed by a thin cut or groove in plate body 152. Each slot 176 includes a linear segment extending to a terminal end 178 located on upper surface 162 between the inner and outer edges 182, 184 of plate body 152. Each slot 176 extends from its terminal end 178 along the linear segment to the sinusoidal shape, and the sinusoidal shape extends from the linear segment across a majority of the width of the respective plate segment to an open side 180 that extends through one of the inner and outer edges 182, 184 of plate body 152.

The edge of plate body 152 through which open end 180 extends alternates between adjacent one of the slots 176 to maintain the structural integrity of plate body 152. For example, two of the slots 176 in lateral portion 154 include open ends 180 that extend through and open at inner edge 182, and the other two slots 176 include open ends 180 that extend through and open at outer edge 184. Each of the slots 176 extends partially into the thickness of plate body 152 from upper surface 162 toward lower surface 164. Other embodiments contemplate that a part of a slot 176 extends completely through upper and lower surfaces 162, 164. Yet other embodiments contemplate locating slots 176 at other portions of plate body 152 and in other patterns and/or densities across plate body 152 to ease contourability of plate body 152. In yet other embodiment, plate body 152 is made from a material that also enhances contourability, such as a flexible fabric, polymer, metal material, or a composite of one or more of these materials in any other number of configurations.

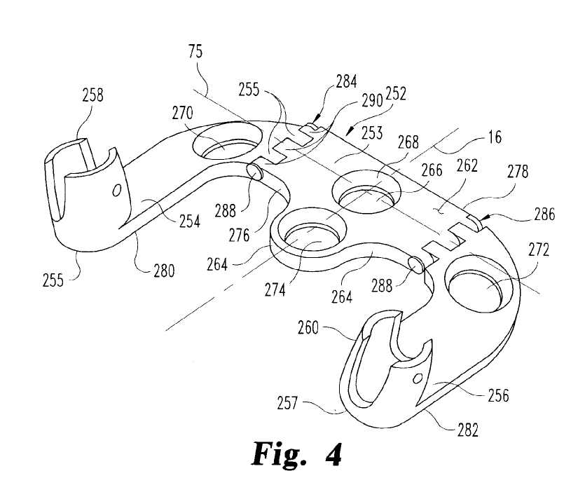

Referring to FIG. 4, there is shown another plate assembly embodiment designated at 250 that is positionable along the occiput 18 in a manner similar to that discussed above with respect to plate assembly 50. Plate assembly 250 includes plate body 252 with coupling members 258, 260 that can include any of the features of coupling member 58, 60 discussed above with respect to plate assembly 50 to engage connecting elements 22, 24. Plate body 252 includes a central portion 253 that extends transversely to the central axis 16 and along medial-lateral axis 75 to opposite lateral portions 254, 256. Lateral portions 254, 256 are located bi-laterally relative to central axis 16 and extend caudally from medial-lateral axis 75 to laterally offset coupling member 258, 260 to opposite sides of the medial nuchal line. Lateral portions 254, 256 extend caudally in a direction generally along central axis 16 to a respective one of the outer ends 255, 257 of plate body 252. In addition, plate body 252 includes a central projecting portion 264 extending from central portion 253 along central axis 16 in a caudal direction.

Plate body 252 includes an upper or proximally-directed surface 262 and an opposite lower or distally directed surface 264. A number of holes, slots or windows 266, 270, 272, 274 are provided through plate body 252, and each hole opens at upper and lower surfaces 262, 264. Each of holes 266, 270, 272, 274 is surrounded by a recessed surface 268 in plate body 252. Recessed surface 268 extends completely around the respective hole and receives a fastener 30 positioned in the respective hole so that the head 32 of fastener 30 is located at least partially in the respective hole and recessed more distally relative to upper surface 262 than if recessed surface 268 were not provided to reduce the profile of the fastener extending from upper surface 262.

Plate body 252 also includes a caudally oriented sidewall 276 extending between upper and lower surfaces 262, 264 and an opposite cephaladly oriented sidewall 278 extending between upper and lower surfaces 262, 264. Caudal sidewall 276 and cephalad sidewall 278 extend across central axis 16 between lateral portions 254, 256. Lateral portions 254, 256 each include a U-shaped sidewall 280, 282, respectively, that extends between and connects sidewalls 276, 278. Sidewalls 276, 278, 280, 282 are configured to provide plate body 252 with a generally M-shaped configuration with the legs of the M pointing in the caudal direction when implanted. Other embodiments contemplate other shapes for plate body 252, including U-shapes, V-shapes, W-shapes, C-shapes, E-shapes, K-shapes, Y-shapes, butterfly shapes, and square or rectangular bracket type shapes, for example.

Plate body 252 also includes coupling mechanisms 284, 286 at opposite ends of central portion 253 that adjustably couple lateral portions 254, 256 to respective ends of central portion 253. Coupling mechanisms 284, 286 each include a coupling member 288 extending through aligned bores of inter-digitated parts of the respective lateral portion 254, 256 and central portion 253. Central portion 253 includes fingers 290 spaced from one another, and lateral portion 254 includes fingers 255 spaced from one another and positioned between fingers 290. Coupling member 288 is in the form of a pin that is positioned through aligned bores of fingers 255, 290 to pivotally couple lateral portion 254 to central portion 253. Lateral portion 256 is similarly coupled to the opposite end of central portion 253.

In the illustrated embodiment, coupling mechanism 284, 286 provide pre-built mechanisms in plate assembly 250 that assist the surgeon in adjusting the contour of plate body 252 to fit the occiput and reduce time in the operating room to bend or curve plate body 252 to fit the patient anatomy. Coupling mechanisms 284, 286 provide rotational or spherical joints at each end of central portion 253 that allow lateral portions to be moved relative to central portion 253 around central axis 16 without bending plate body 252. Other embodiments contemplate that only one coupling mechanism is provided, or that more than two coupling mechanisms are provided to provide more than two adjustment locations for plate body 252.

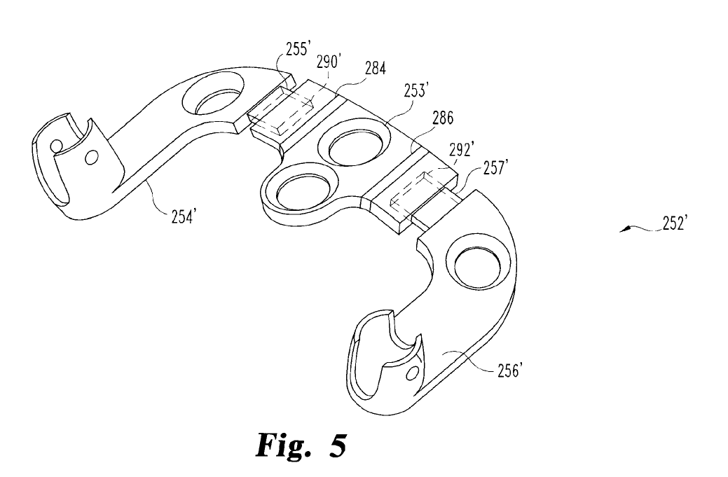

FIG. 5 shows another embodiment plate assembly 250′ that is similar to plate assembly embodiment 250, but also incorporates lateral adjustability of lateral portions 254′, 256′ in addition to the contouring adjustability of plate body 252 provided in the FIG. 4 embodiment. Plate body 252′ includes coupling mechanism 284, 286 shown diagrammatically, adjacent opposite ends of central portion 253′. Central portion 253′ also includes opposite end openings 290′, 292′ extending laterally away from the adjacent coupling mechanism 284, 286 to receive an extension 255′, 257′ from a respective one of the lateral portions 254′, 256′. Extensions 255′, 257′ are translatable in respective ones of the end openings 290′, 292′ to provide medial-lateral adjustment in the width of plate body 252′, facilitating positioning of plate body 252′ and lateral portions 254′, 256′ in the desired and optimal location in which to fit the patient anatomy and to engage fasteners 30 to the occiput 18. In other embodiments, one or both of coupling mechanisms 284, 286 are provided on the respective lateral portion 254′, 256′.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character. All changes and modifications that come within the spirit of the invention are desired to be protected.

What is claimed is:

1. An occipital plating system, comprising:
a plate body including a central portion extending along a medial-lateral axis and opposite lateral portions extending from opposite ends of said central portion, said opposite lateral portions extending along a central axis transversely to said medial lateral axis, said plate body further including an upper surface and a lower surface opposite said upper surface, said lower surface for positioning distally toward an occiput, said lateral portions each including a coupling member associated therewith and projecting outwardly from said plate body, said coupling members each configured to receive a connecting element extending from a vertebra to the occiput when the plate is positioned on the occiput, said central portion and said lateral portions extending around a common window extending through said upper and lower surfaces to receive at least one fastener through said window.

2. The plating system of claim 1, wherein said plate body includes a central projecting portion extending from said central portion along said central axis and said common window extends through said central projecting portion.

3. The plating system of claim 2, wherein said window includes a central leg extending along said central portion, lateral legs extending transversely to said central leg along said lateral portions, and a middle leg extending transversely to said central leg along said central projecting portion.

4. The plating system of claim 3, wherein said window is continuous between said central leg, said middle leg, and said lateral legs.

5. The plating system of claim 4, wherein said plate body includes a recessed surface extending into said upper surface and said recessed surface extends completely around said window.

6. The plating system of claim 4, wherein said plate body and said window form an E-shape when viewed from a direction looking toward said upper surface of said plate body.

7. An occipital plating system, comprising:
a plate body for positioning on the occiput, the plate body including a central portion extending along a medial-lateral axis and opposite first and second lateral portions extending from opposite ends of said central portion and along a central axis and transversely to said medial lateral axis, said lateral portions each including a coupling member associated therewith and projecting outwardly from said plate body, said coupling members configured to receive an elongated connecting element extending from a vertebrae to the occiput when the plate is positioned on the occiput, said plate body further including:
an upper surface and a lower surface opposite said upper surface, said lower surface for positioning distally toward the occiput;
an inner edge and an opposite outer edge, said inner and outer edges extending between said upper and lower surfaces, said inner edge being located along facing sides of said first and second lateral portions and along a first side of said central portion that extends between said facing sides, and said outer edge is located along said first and second lateral portions and said central portion opposite said facing sides of said first and second lateral portions and said first side of said central portion;
a first plurality of slots in said first lateral portion and a second plurality of slots in said second lateral portion, said first and second plurality of slots extending across said first and second lateral portions in a direction generally along said medial-lateral axis; and
a third plurality of slots extending across said central portion in a direction generally along said central axis.

8. The plating system of claim 7, wherein each of said slots includes an alternating shape across said upper surface of said plating member.

9. The plating system of claim 8, wherein each of said slots includes a terminal end located in said plate body between said inner and outer edges, each of said slots extending from said terminal end to an open end that extends through one of said inner and outer edges.

10. The plating system of claim 9, wherein adjacent ones of said slots are oriented so that one of said adjacent slots includes said open end thereof extending through said inner edge and the other of said adjacent slots includes said open end thereof extending through said outer edge.

11. The plating system of claim 10, wherein said adjacent slots are configured so that peaks of said alternating shape of one of said adjacent slots are generally aligned with respective peaks of said alternating shape of the other of said adjacent slots.

12. The plating system of claim 11, wherein said first and second plurality of slots include a spacing between a most-caudal slot and a most-cephalad slot thereof that extends along a majority of a length of respective ones of said first and second lateral portions and said third plurality of slots include a spacing between most-lateral ones of slots thereof that extends along a majority of a length of said central portion.

13. The plating system of claim 7, wherein said plate body includes a first hole on said central axis opening at said upper and lower surfaces and said third plurality of slots include slots located on opposite sides of said first hole.

14. The plating system of claim 13, wherein said plate body includes a second hole opening at said upper and lower surfaces between said first plurality of slots and said third plurality of slots and said plate body includes a third hole opening at said upper and lower surfaces between said second plurality of slots and said third plurality of slots.

15. The plating system of claim 7, wherein each of slots extends across a majority of a width of said plate body between said inner and outer edges of said plate body.

16. An occipital plating system, comprising:
a plate body including a central portion extending along a medial-lateral axis and opposite first and second lateral portions extending from opposite ends of said central portion, said first and second lateral portions extending from said central portion along a central axis transversely to said medial lateral axis, said first and second lateral portions each including a coupling member associated therewith and projecting outwardly from said plate body, said coupling members each being configured to receive a connecting element extending from a vertebrae to an occiput when the plate is positioned on the occiput, said plate body further including:
a first coupling mechanism pivotably coupling said first lateral portion with said central portion; and
a second coupling mechanism pivotable coupling said second lateral portion with said central portion, wherein said first and second coupling mechanisms allow said first and second lateral portions to rotate relative to said central portion around said central axis.

17. The plating system of claim 16, wherein each of said first and second lateral portions includes an extension extending along said medial lateral axis and said central portion includes first and second receptacles opening at opposite ends of said central portion, said extensions being adjustably received in respective ones of said receptacles to allow said first and second lateral portions to be moved toward and away from said central axis along said medial-lateral axis.

18. The plating system of claim 17, wherein said first coupling mechanism is located between said first receptacle and said central axis and said second coupling mechanism is located between said second receptacle and said central axis.

19. The plating system of claim 16, wherein each of said first and second coupling mechanisms includes:
a number of fingers extending from said central portion positioned adjacent a number of fingers extending from a respective one of said first and second lateral portions and a pin extending through said fingers, said pin defining a rotation axis that generally parallels said central axis.
